SEQUENCE LISTING (4 sequences)

SEQ ID NO: 1 - DNA, 3334 bp, Artificial Sequence
  Synthetic Primer
actttgagtactgtgaggtcacaaaccacatgattctgtctctccagtaatagtgcttgcaaaaaataggagttttaaagcttttgcttttttggattgtgtgaatgcttcattcgcctcacaaacaaccacagaaccacaagtgcggtgcaaactttctccaggaagactgcaagaaggctctggcgtttaaatggttaatctctgcaggtcactaccagccaccgagaccaaccgagtcatttaaggctgcaagcagtatttacaacagagggcacaagttctatctggaaaaaaaaggagggactatggcgtcaaacagcctcttcagcgcagtgacaccgtgtcagcaaagcttcttttgggatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgttttttttcttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggagcggggagggtggccattatgactcttatcatattaaaaagccaatgcacaagtgattggttgaactgcagaaaagtgttctgtggtctctgagttgagcaaaactctaaattgcaggcttcgtggttgagggcctagtcagctgaaagccacgcgt

SEQ ID NO: 2 - protein, 596 aa, Artificial Sequence
  Synthetic Peptide
MLHSPHKQPQNHKCGANFLQEDCKKALAFKWLISAGHYQPPRPTESFKAASSIYNRGHKFYLEKKGGTMASNSLFSAVTPCQQSFFWDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMGYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 3 - DNA, 2294 bp, Artificial Sequence
  Synthetic Peptide
atggcgtcaaacagcctcttcagcgcagtgacaccgtgtcagcaaagcttcttttgggatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagattcagaacccagcaccttggactcacagtcttccaccaccctgttcctgtctccagaggagcctggcccctctacagcagctctgccatctccatcctcgtcctgtgagccccagcccttctctcccagccccatgttgccccctctcctgcagcctctgtccactgcctccacagtgccagccccctgcgtccgtcggcgcactgggctctacaccattgtgacctcctccccagaggctgcaccccaccttgttgactggatgcccagctgccccactgccacgtcccctggtgtccgaggcaaggatcatgagcggccacagaccatgatggccccggccccagctctagcttcagagaggggccacagtcagcatgcaggccctgccagggatgatcatgctgaacatcctggaacctccccaaagccctgtgctcctccagccgctgctgccaccttggaggccagtgttggggacatcctggtggagctacggacaatgaatggccatctggacatcatagcaaaggccctcactaaattggcctcttctctggtgccccagtctcagcctgtgcctgaagcaccagatgccaattaaaagagctggacttctaaacaaagggatgctgaggtaccacacatcctcgctgactcctctgatcccgtctttgctggagacaagcaaaatcagcagaagagccagttacttagcttggtttgctcacacattggatgcccttggcctgtcactcaaaggatagcagtgtcctgctggctccacgagctgacaagctgtagactttctgtgttccctttcaccttccaatgcccctctctcgttctaaatccccaatagaagaagcaccccgagaagttccagggacaaggtgattggaaaggtatcattccctcccagtccagcgaacccccagccaagcgagcggaggggagaggaggtcctatcgtcagaaacactttctggggcccctccagcatccctttcttgagatgctgatggtgttgccacctccagtggactccagtggacttcacatatctcttcttaaagtctctttaggaaaaccaaattgattgtttttctcatttatacctacatttcaaagagtttgggaaataggcaaggagggaaaaggacagagcagaaagacggtgggggctatttaggtctttcttatgaataagagtatcatgtgtcctgataaagtgtgtgtctatactctctga

SEQ ID NO: 4 - protein, 548 aa, Artificial Sequence
  Synthetic Peptide
MASNSLFSAVTPCQQSFFWDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDSEPSTLDSQSSTTLFLSPEEPGPSTAALPSPSSSCEPQPFSPSPMLPPLLQPLSTASTVPAPCVRRRTGLYTIVTSSPEAAPHLVDWMPSCPTATSPGVRGKDHERPQTMMAPAPALASERGHSQHAGPARDDHAEHPGTSPKPCAPPAAAATLEASVGDILVELRTMNGHLDIIAKALTKLASSLVPQSQPVPEAPDAN

(19) United States
(12) Patent Application Publication
(10) Pub. No.: US 2003/0235564 A1
(43) Pub. Date: Dec. 25, 2003

(54) Compositions and devices comprising or encoding the Run x2 protein and method of use

(76) Inventors: Bruce Doll, Wexford, PA (US); Huihua Fu, Pittsburgh, PA (US); Jeffrey O. Hollinger, Gibsonia, PA (US); Charles Sfeir, Pittsburgh, PA (US)

Correspondence Address: MCANDREWS HELD & MALLOY, LTD, 500 WEST MADISON STREET, SUITE 3400, CHICAGO, IL, 60661

(21) Appl. No.: 10/437,171

(22) Filed: May 13, 2003

Related U.S. Application Data
(60) Provisional application No. 60/380,554, filed on May 13, 2002.

(51) Int. Cl. A61K 48/00; A61K 38/17

(52) U.S. Cl. 424/93.21; 424/426; 514/12; 514/44

(57) ABSTRACT

A pharmaceutical composition comprising in combination the Runx2 protein, a polynucleotide encoding the Runx2 protein, or a cell that has been transformed with a polynucleotide encoding Runx2 protein, in a pharmaceutically acceptable carrier, the carrier comprising a bio-compatible, biodegradable polymeric matrix. Another aspect of the present invention includes a device comprising the above-described pharmaceutical composition in combination with a sterile and substantially antigen-free, pre-shaped allograft or xenograft bone implant. Also contemplated herein is a method for repairing a bone defect comprising administering to a mammalian patient at the site in need of treatment a pharmaceutical composition, comprising in combination the Runx2 protein, a polynucleotide encoding the Runx2 protein, or a cell that has been transformed with a polynucleotide encoding Runx2 protein, in a pharmaceutically acceptable carrier wherein the carrier is a bio-compatible, biodegradable polymeric matrix.

### RELATED APPLICATIONS

This application is related to U.S. Provisional Application Serial No. 60/380,554, filed May, 13, 2002.

### FEDERALLY SPONSORED RESEARCH OR DEVELOPMENT

The research underlying this application was supported in part by federal funding from The National Institute of Standards and Technology (NIST), Award No. 70NANB1H3060, granted to Regeneration Technologies, Inc., such that the Federal government may have some rights in the presently disclosed invention

### MICROFICHE/COPYRIGHT REFERENCE

[Not Applicable]

### BACKGROUND OF THE INVENTION

It is estimated 33 million individuals per year who sustain musculoskeletal injuries and require some type of bone grafting procedure. Predictable, successful outcome with bone grafts, especially autografts, sustains popularity. However, for patients who are medically compromised by pathphysiological status and receive autogenous bone grafts, the success rate decreases. Conventional therapies of autografting and grafting of allogeneic-banked bone can be clinically adequate. However, there are recognized limitations to these therapies that may be addressed with alternative treatments. Alternatives have included bone-graft substitutes, which may contain soluble signaling molecules, such as morphogens and/or mitogens to further enhance the regenerative process. Furthermore, recent developments in cell and molecular biology have enabled researchers in the bone tissue-engineering field to develop new strategies for gene and cell-based therapies.

Local gene therapy treatments may be options to compensate for a high-dose-for-effect of growth factor at an osseous wound site. A collagen carrier containing DNA plasmids encoding human PTH 1-34 and/or mouse BMP-4 have exhibited promising in situ delivery with pre-clinical results for regenerating skeletal defects. Several ex vivo approaches have included adenoviral gene transfer to generate BMP-2-producing bone-marrow cells. For these studies, critical-sized defects in rats were treated with genetically modified autogenous marrow cells delivered by allogeneic, inactivated demineralized bone matrix. Although successful at regenerating the defects, there were concerns about safety of the adenoviral vector, the immunogenic impact from both from the vector and the allogeneic delivery system, and the fate of BMP-transfected cells.

Recombinant human bone morphogenetic proteins (rhBMPs) have aroused considerable enthusiasm from clinicians. However, only three peer-reviewed clinical studies using rhBMP have been published and BMP doses suggesting efficacy ranged from 1.7-3.4 mgs. Dosing patients with high levels of BMP, which is a potent morphogen, mutes clinical enthusiasm. Other growth factors have stimulated interest (for example, insulin-like growth factor (IGF), platelet-derived growth factor (PDGF), and fibroblast growth factor-2 (FGF-2)) without predictable results. Difficulties promoting a clinical effect with growth factors may be due to insufficient dosing and temporal inconsistencies; asynchrony between kinetics of signaling receptor expression and growth factor availability; transient active half-life of growth factors (i.e., minutes); diffusivity; sequestration by plasma membranes; lysis in the wound healing environment; or an inadequate responding cell population. To overcome these difficulties using growth factors, alternatives have been investigated.

An alternative ex vivo method of local gene therapy involved cultured periosteal cells transduced retrovirally with BMP-7 and delivered with poly(glycolic) acid (PLGA) to restore critical-sized defects in the calvaria of rabbits. Retroviral vectors, which are integrating vectors, may be superior to adenoviral in cases where long term expression is required such as to treat hereditary or chronic disorders. However, during integration of the retroviral proviral DNA, insertional mutagenesis can occur into the targeted genome if the inserted DNA disrupts housekeeping genes or activates other genes, e.g., an oncogene.

The present use of pluripotent growth factors, BMPs etc, offers relatively less focus on the cell types and intended outcome. Potential effects of induced non-osteogenic activity are a risk with factors known to have various actions on different cell types. Thus, a more focused approach to stimulating osteoblastic activity would be desirable in inducing bone healing.

### BRIEF SUMMARY OF THE INVENTION

The Applicants have discovered that tissue engineered constructs comprising the Runx2 protein [e.g., SEQ ID NOs: 2 and 4], polynucleotides [SEQ ID NOs: 1 and 3] encoding Runx2 protein, and cells that have been transformed with a polynucleotide encoding Runx2 protein in provide a focused approach to the repair of bone via bone induction.

Accordingly, the present invention has multiple aspects. In its first aspect, the present invention is directed to a pharmaceutical composition comprising in combination the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. Preferably, the carrier is a bio-compatible, polymeric matrix that is slowly decomposed (over 1-3 months, preferably 2-3 months) and absorbed by the body so as to temporarily release the Runx2 protein locally in vivo at the site where bone repair and/or osteoblastic activity is needed. Typical biocompatible polymers include synthetic polymers such as the hydrogel forming acrylates and methacrylates, and naturally occurring polymers such as hydrated collagen or atelocollagen in thermoplastic form. In another embodiment, the present invention is directed to a pharmaceutical composition comprising in combination a sterile and substantially antigen-free allograft bone implant, and the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier.

In its second embodiment, the present invention is directed to a medical device comprising in combination a sterile and substantially antigen-free, pre-shaped allograft bone implant, and the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. Preferably, the allograft bone implant has one or more cavities (typically, one cavity) formed therein for receiving the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. In another embodiment the allograft bone implant is partially demineralized. In yet another embodiment, the allograft bone implant is a composite device composed of three or more pieces of sterile and substantially antigen-free, pre-shaped allograft bone. In the latter embodiment, it is preferred that one of the pieces of sterile and substantially antigen-free, pre-shaped allograft bone be a piece of cancellous bone.

In its third aspect, the present invention is directed to a method for repairing a bone defect comprising administering to a mammalian patient at the site in need of treatment a pharmaceutical composition, comprising in combination the Runx2 protein [e.g., SEQ ID NOs: 2 or 4], a polynucleotide [e.g., SEQ ID NOs: 1 or 3] encoding the Runx2 protein, or a cell that has been transformed with a polynucleotide encoding Runx2 protein, in a pharmaceutically acceptable carrier. Preferably, the carrier is a bio-compatible, polymeric matrix that is slowly decomposed and absorbed by the body so as to temporarily release the Runx2 protein locally in vivo at the site where bone repair and/or osteoblastic activity is needed. In another embodiment, the present invention is directed to a method for repairing a bone defect comprising administering to a mammalian patient at the site in need of treatment a sterile and substantially antigen-free, pre-shaped allograft bone implant sized and shaped to fit the bone defect and a pharmaceutical composition, comprising in combination the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. In the method of the present invention, the pharmaceutical composition comprising the Runx2 protein (or encoding polynucleotide or transformed cell) and its carrier, is combined with the sterile and substantially antigen-free, pre-shaped allograft bone implant within two hours of implantation into the mammalian patient, preferably within one hour, more preferably within one-half hour prior to implantation. Typically, the mammalian patient of the present method is a human patient or a domesticated animal, such as a dog, cat, horse, bovine animal, or porcine animal. Preferably, the mammalian patient of the present method is a human patient.

It is also within the scope of the present invention that the term “Runx2” includes those proteins that have one or more conservative amino acid substitutions relative to the amino acid sequence disclosed in SEQ ID NO: 2 or 4. Those skilled in the art recognize that conservative amino acid substitutions would not be expected to affect the biological activity of the Runx2 protein. Preferably, the term “Runx2” encompasses those amino acid sequences that have 95% sequence identity with the amino acid sequence disclosed in SEQ ID NO: 2 or 4, more preferably 95% sequence identity. It is also within the scope of the present invention that the tern “Runx2” encompasses those proteins of that SEQ ID NO: 2 or 4 that have a truncation of up to 5% of the amino acids at the carboxy terminus.

The term “polynucleotide encoding Runx2” and its synonyms is intended to encompass the nucleotide sequences of SEQ ID NOs 1 or 3, and includes the substitution of codons that encode for the same amino acid.

### BRIEF DESCRIPTION OF SEVERAL VIEWS OF THE DRAWINGS

[Not Applicable]

### DETAILED DESCRIPTION OF THE INVENTION

The present invention has multiple aspects. In its first aspect, the present invention is directed to a pharmaceutical composition comprising in combination the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. Preferably, the carrier is a bio-compatible, polymeric matrix that is slowly decomposed and absorbed by the body so as to temporarily release the Runx2 protein locally in vivo at the site where bone repair and/or osteoblastic activity is needed. Typical biocompatible polymers are described in detail herein but include synthetic polymers such as the hydrogel forming acrylates and methacrylates, and naturally occurring polymers such as hydrated collagen or atelocollagen in thermoplastic form. In another embodiment, the present invention is directed to a pharmaceutical composition comprising in combination a sterile and substantially antigen-free allograft bone implant, and the Runx2 protein [e.g., SEQ ID NOs: 2 or 4], a polynucleotide [SEQ ID NOs: 1 or 3] encoding the Runx2 protein, or a cell that has been transformed with a polynucleotide encoding Runx2 protein, in a pharmaceutically acceptable carrier. A method for making a sterile and substantially antigen-free allograft bone implant is disclosed in commonly assigned U.S. Pat. No. 6,428,584, which issued to Mills et al. on Nov. 19, 2002, and which is entitled “Cyclic Implant Perfusion Cleaning and Passivation Process.”

In its second embodiment, the present invention is directed to a medical device comprising in combination a sterile and substantially antigen-free, pre-shaped allograft or xenograft bone implant, and the Runx2 protein [e.g., SEQ ID NOs: 2 and 4], a polynucleotide [SEQ ID NOs: 1 and 3] encoding the Runx2 protein, or a cell that has been transformed with a polynucleotide encoding Runx2 protein, in a pharmaceutically acceptable carrier. Preferably, the bone implant is an allograft. More preferably, the bone implant has one or more cavities (typically, one cavity) formed therein for receiving the Runx2 protein [e.g., SEQ ID NOs: 2 and 4], a polynucleotide [SEQ ID NOs: 1 and 3] encoding the Runx2 protein, or a cell that has been transformed with a polynucleotide encoding Runx2 protein, in a pharmaceutically acceptable carrier. Examples of suitable implants having one or more cavities for receiving the Runx2 gene is disclosed in the following U.S. Patents: U.S. Pat. No. 6,096,081, which issued to Grivas et al. on Aug. 1, 2000, and which is entitled “Diaphysial Cortical Dowel;” U.S. Pat. No. 6,290,718, which issued to Grooms, et al. on Sep. 18, 2001, and which is entitled “Luminal Graft, Stent or Conduit Made of Cortical Bone;” U.S. Pat. No. 6,409,765, which issued to Bianchi, et al. on Jun. 25, 2002, and which is entitled “Open Intervertebral Spacer;” and in the following commonly assigned U.S. patent applications: U.S. Ser. No. 10/375,540, entitled “Cortical Bone-based Composite Implants,” now-pending, filed Feb. 27, 2003 and claiming priority to Aug. 27, 1997; and U.S. Ser. No. 09/782,594, entitled “Assembled Implant,” now pending, and filed Feb. 12, 2001 and claiming priority to Feb. 10, 2000, all of which are hereby incorporated by reference in their entirety. In another embodiment, the allograft bone implant is partially demineralized. Methods for the demineralization of cortical and cancellous bone are well known in the art, such as disclosed in Bowman, et al., “The tensile behavior of demineralized bovine cortical bone,” J. Biomech, 29(11):1497-1501 (1996); Broz, et al., “Material and compositional properties of selectively demineralized cortical bone,” J. Biomech, 28(11):1358-68 (1995); and Rosenthal et al., “Demineralized bone implants for nonunion fractures, bone cysts, and fibrous lesions,” Clin Orthop, 364:61-69 (1999), all of which are incorporated herein for their disclosures on the preparation of demineralized bone.

In yet another embodiment of the above-described medical device, the allograft bone implant is a composite device composed of three or more pieces of sterile and substantially antigen-free, pre-shaped allograft or xenograft bone. Methods for making composite devices out of allograft and/or xenograft bone are disclosed in commonly assigned U.S. patent applications: U.S. Ser. No. 10/375,540, entitled “Cortical Bone-based Composite Implants,” now-pending, filed Feb. 27, 2003 and claiming priority back to Aug. 27, 1997; and U.S. Ser. No. 09/782,594, entitled “Assembled Implant,” now pending, filed Feb. 12, 2001 and claiming priority to Feb. 10, 2000; and in U.S. Pat. No. 6,200,347, which issued to Anderson et al. on Mar. 13, 2001 and which is entitled “Composite Bone Graft, Method of Making and Using Same,” all of which are incorporated herein by reference in their entirety. In the latter embodiment, it is preferred that at least one of the pieces of sterile and substantially antigen-free, pre-shaped allograft (or xenograft) bone be a piece of cancellous bone. Preferably, the allograft bone implant is an allograft bone implant.

In its third aspect, the present invention is directed to a method for repairing a bone defect comprising administering to a mammalian patient at the site in need of treatment a pharmaceutical composition, comprising in combination the Runx2 protein [e.g., SEQ ID NOs: 2 and 4], a polynucleotide [SEQ ID NOs: 1 and 3] encoding the Runx2 protein, or a cell that has been transformed with a polynucleotide encoding Runx2 protein, in a pharmaceutically acceptable carrier. Preferably, the carrier is a bio-compatible, polymeric matrix that is slowly decomposed and absorbed by the body so as to temporarily release the Runx2 protein locally in vivo at the site where bone repair and/or osteoblastic activity is needed. In another embodiment, the present invention is directed to a method for repairing a bone defect comprising administering to a mammalian patient at the site in need of treatment a sterile and substantially antigen-free, pre-shaped allograft bone implant sized and shaped to fit the bone defect and a pharmaceutical composition, comprising in combination the Runx2 protein [e.g., SEQ ID NOs: 2 and 4], a polynucleotide [SEQ ID NOs: 1 and 3] encoding the Runx2 protein, or a cell that has been transformed with a polynucleotide encoding Runx2 protein, in a pharmaceutically acceptable carrier. In the method of the present invention, the pharmaceutical composition comprising the Runx2 protein (or encoding polynucleotide or transformed cell) and its carrier, is combined with the sterile and substantially antigen-free, pre-shaped allograft bone implant within two hours of implantation into the mammalian patient, preferably within one hour, more preferably within one-half hour prior to implantation. Typically, the mammalian patient of the present method is a human patient or a domesticated animal, such as a dog, cat, horse, bovine animal, or porcine animal. Preferably, the mammalian patient of the present method is a human patient.

In the first aspect described above, the present invention is directed to a pharmaceutical composition comprising the nuclear transcription factor Runx2 engineered with several possible types of delivery systems. The delivery systems comprising in combination Runx2 protein (or a nucleotide encoding Runx2 protein) in a polymeric carrier provides bone regeneration therapy that produces a predictable bone formation outcome in patients. The patients who would benefit from the composition of Runx2 and delivery system include patients with bone insufficiencies caused by malformation, disease, and trauma. Moreover, the composition enhances the rate of bone formation for healthy patients. To the extent that the Applicants cite to the prior art herein for conventional methods and compositions, all publication and patents of any type that are cited anywhere in this document are expressly incorporated herein by reference in their entirety.

Runx2

Recent discoveries have revealed the transcription factor, Runx2, also referred to as Cbfa1 (core binding factor alpha 1) and as Osf2 (osteoblast specific factor 2), which is a regulator of osteoblast differentiation. See U.S. Pat. No. 6,518,063, which issued to Ducy et al. on Feb. 11, 2003 and is entitled “Osf2/Cbfa1 nucleic acids and methods of use therefor.” There are three names in current use for each of the three-mammalian genes encoding the alpha subunit. An attempt to simplify the nomenclature is being made. The gene designation Runx is currently favored for this family of genes by the majority of the core committee. Accordingly, the name “Runx2” is used in the application to refer to the gene and “Runx2” is used to refer to the protein and to the nucleic acid sequence encoding the protein.

Runx2 is the mouse homologue of the Drosophila pair rule gene runt exhibiting 66% identity. Alternative use of transcription start sites and splicing leads to the genesis of isoforms with possible differences in transactivation potentials; however there is remarkable sequence homology for the runt domain across species. Conservation enables cross species cell transformation. The expression of Runx2 in the mouse is primarily limited to regions of mesenchymal condensation that form the future skeleton. Runx2 is the only osteoblast specific transcription factor identified to date. Molecular and genetic evidence have demonstrated that it acts as an activator of osteoblast differentiation during embryonic development in mouse and humans. Indeed, Runx2 is expressed in cells of the osteoblastic lineage during development; it regulates osteoblast-specific expression of the genes for osteocalcin and osteopontin. The promoter of the osteocalcin gene is active only in differentiated osteoblasts able to produce a matrix. Runx2 can induce osteoblastic differentiation of non-osteoblastic cells, and patients heterozygous for mutations or deletions of Runx2 develop cleidocranial dysplasia (CCD). Likewise, inactivation of Runx2 in mice leads to a total absence of osteoblasts in homozygous mutant animals, and to a CCD phenotype in heterozygous mutant animals. Thus, Runx2 is an indispensable regulator of osteoblast differentiation that fulfills a function dominant to and non-redundant with the function of any other gene product. This progress in our understanding of osteoblast differentiation during development is remarkable. Knowledge about the regulation of bone formation supports a role for Runx2.

Two complementary sets of data suggest that Runx2 may be involved in postnatal bone formation. First, Runx2 is expressed at high levels in osteoblasts after birth. Second, it regulates the in vitro and in vivo expression of the gene for osteocalcin, a gene essentially not expressed before birth and that is the hallmark of the differentiated osteoblast phenotype. Ducy and coworkers tested whether Runx2 is a determinant of bone formation by differentiated osteoblasts postnatally. The observation that haploinsufficiency at the Runx2 locus causes CCD suggests that if Runx2 controls bone formation postnatally it should be possible to demonstrate this by altering its level of expression and/or its function postnatally. The availability of a cell-specific and stage specific promoter, such as the promoter of the osteocalcin gene that is not active before birth, provided an excellent tool to evaluate the role of Runx2. Runx2 is required for bone formation by differentiated osteoblasts after birth. By controlling its own expression positively, Runx2 is at the top of a genetic cascade regulating bone extracellular matrix (ECM) deposition. Inhibition of this autoregulatory loop in differentiated osteoblasts resulted in an osteopenic phenotype caused by the near abolition of expression of ECM-related genes, including type I collagen-encoding genes, without any overt effect on osteoblast differentiation. Runx2 appears to be the first transcriptional activator in a transcriptional pathway governing bone formation by differentiated osteoblasts.

Runx2 is one of the three known mouse Cbfa genes. In Western blot analysis, an antiserum that recognizes all three Cbfa proteins detected only Runx2 in calvaria osteoblasts thus indicating that Runx2 is the only Cbfa protein detectable in osteoblasts, which is consistent with the phenotype observed when the gene is deleted. Genetic studies have identified the lack of expression of one allele of Runx2 as the basis for the cleidocranial dysplasia (CCD) in mice and humans. CCD is an autosomal dominant inherited disorder characterized by delayed ossification, patent fontanelles and wormian bones.

Osteoinductive factors like the BMPs (bone morphogenetic proteins) regulate Runx2 expression in osteoblasts. However, Runx2 is different from other molecules such as the bone morphogenetic proteins (BMPs) that can induce bone formation by recapitulating all the cell differentiation events occurring during skeleton development. Instead, Runx2 acts as a maintenance factor of the differentiated osteoblasts by simply regulating the rate of bone matrix deposition by already differentiated cells.

Runx2 function has important biological implications. The transcriptional molecule is able to regulate the expression of multiple extracellular matrix genes in osteoblasts and its over expression can induce osteoblast-specific gene expression in fibroblasts and myoblasts. Mice, homozygous for a targeted deletion of Runx2, showed no osteoblast differentiation and hence completely lack bone. In conclusion, Runx2 is a transcriptional activator of osteoblast differentiation and is needed for in vivo bone formation.

Mutations in the Runx2 gene that leave intact the DNA-binding domain of Runx2 but affect its transactivation function may result in slightly more severe postnatal manifestations in CCD patients. More generally, mutations affecting one of the transactivation domains of Runx2 could be at the origin of some form of genetically inherited low or high bone mass diseases. The ability of Runx2 to control the function of already differentiated osteoblasts, together with the fact that haploinsufficiency at the Runx2 locus is at the origin of CCD suggests that increasing Runx2 rate of transcription in elder individuals may be a way to prevent or to treat osteopenic diseases and skeletal defects resulting from trauma or a typical development. Runx2 is a tool for the enhanced bone generation by encouraging specific cellular responses culminating in bone formation.

Delivery System

Bone graft substitute materials for the musculoskeletal system are designed to mimic many of the components found in the intact, healthy bone. These components include a biologic porous substratum (referred to as a matrix, carrier, delivery system, and scaffold), extracellular matrix, cells, and signaling molecules. The laboratory-derived composition has therefore been called a tissue engineered “bone biomimetic”. The bone biomimetic should provide an architectural environment conducive for the regeneration and ingrowth of osseous tissue at the site of injury.

In terms of scaffolds, clinical progress to date has primarily emphasized suitable scaffolds incorporating an osteoinductive class of signaling molecules known as the bone morphogenetic proteins (BMPs) to regenerate bone. Biomimetic scaffolds that incorporate signaling molecules, genes, cells, or genetically modified cells as a tissue-engineered construct, offer a platform as a musculoskeletal delivery system.

The delivery system must be engineered to permit the ingrowth of osseous tissue at the site of injury and should be capable of delivering signaling molecules, genetic materials or cells, together or individually, to the surgical (implant) site. The delivery system must be surgically convenient, remain localized to the implant site, and provide the roles of substratum, as well as a carrier. A carrier's geometric, mechanical, and chemical properties can be engineered to ensure optimal host cell invasiveness to populate and mineralize the scaffold (substratum). Furthermore, the scaffold/carrier should exhibit formability and ease of use. Safety issues related to method for sterilization should not impact the previously described characteristics or biocompatibility, and should not impair sustained release delivery capabilities. The carrier can localize, protect and present plasmid DNA or other biologics in a sustained manner to impact the bone regenerative cascade.

Suitable biocompatible polymers for use in the composition, device and method of the present invention include poly(alpha-hydroxy) acids, e.g., poly(lactide) (PLA), and poly(lactide-co-glycolide) (PLGA), as well as collagens (primarily an atelopeptide, type I bovine collagen). Atelocollagen is obtained by treating collagen with pepsin, which removes antigenic telopeptides, responsible for intermolecular cross linkage of collagen. In addition, process modifications for the biocompatible polymers permit the fabrication of a number of configurations, such as a gel, a lyophile or a sheet. The collagen delivery systems, a type I collagen sol/gel, have incorporated BMP growth factors in the past and are processed to obtain a gel, a lyophile or a sheet configuration. Each type of structure provides a different type of sustained release profile that retains the biologic activity of the incorporated run x2 protein, or nucleotide encoding the run x2 protein, or transformed cell having operatively functioning therein a nucleotide encoding the run x2 protein.

Polymers for Formation of Matrices

Over the last decade there has been a tremendous increase in applications for polymeric materials. These materials are well suited to implantation as they can serve as a temporary scaffold to be replaced by host tissue, degraded by hydrolysis to non-toxic products, and be excreted, as described by Kulkarni, et al., J. Biomedical Materials Research, 5, 169-81 (1971); Hollinger, J. O. and G. C. Battistone, “Biodegradable Bone Repair Materials,” Clinical Orthopedics and Related Research, 207: 290-305 (1986), which is incorporated herein by reference.

Either natural or synthetic polymers can be used to form the matrix, although synthetic polymers are preferred for reproducibility and controlled release kinetics. Synthetic polymers that can be used include bioerodible polymers such as poly(lactide) (PLA), poly(glycolic acid) (PGA), poly(lactide-co-glycolide) (PLGA), and other poly(alpha-hydroxy acids), poly(caprolactone), polycarbonates, polyamides, polyanhydrides, polyamino acids, polyortho esters, polyacetals, polycyanoacrylates and degradable polyurethanes, and non-erodible polymers such as polyacrylate, ethylene-vinyl acetate polymer and other acyl substituted cellulose acetates and derivatives thereof, non-erodible polyurethanes, polystyrenes, polyvinyl chloride, polyvinyl fluoride, poly(vinyl imidazole), chlorosulphonated polyolefins, polyethylene oxide, polyvinyl alcohol, and nylon. Although non-degradable materials can be used to form the matrix or a portion of the matrix, they are not preferred. Examples of natural polymers suitable for use in the pharmaceutical composition, device and method of the present invention include proteins, such as albumin, collagen, synthetic polyamino acids, and prolamines; and polysaccharides, such as alginate, heparin, and other naturally occurring biodegradable polymers of sugar units.

Four synthetic biocompatible polymers that are suitable for use in the present invention are poly(paradioxanone) (PDS), poly(lactic acid) (PLA), poly(glycolic acid) (PGA), and PLAGA copolymers. Copolymerization enables modulation of the degradation time of the material. By changing the ratios of crystalline to amorphous polymers during polymerization, properties of the resulting material can be altered to suit the needs of the application. These polymers, including poly(lactide-co-glycolic) acid (PLGA), have been used as polymer composites for bone replacement as reported by H. M. Elgendy, et al. “Osteoblast-like cell (MC3T3-E1) proliferation on bioerodible polymers: An approach towards the development of a bone-bioerodible polymer composite material,” Biomaterials, 14, 263-269 (1993). Substituted polyphosphazenes have been shown to support osteogenic cell growth, as reported by C. T. Laurencin, et al. “Use of polyphosphazenes for skeletal tissue regeneration,” J. Biom. Mater. Res., 27 (1993). Poly(organophosphazenes) are high molecular weight polymers containing a backbone of alternating phosphorus and nitrogen atoms. There are a wide variety of polyphosphazenes, each derived from the same precursor polymer, poly(dichlorophosphazene). The chlorine-substituted species can be modified by replacement of the chlorine atoms by different organic nucleophiles such as o-methylphenoxide along with amino acids. The physical and chemical properties of the polymer can be altered by adding various ratios of hydrolytic sensitive side chains such as ethyl glycinate, as described by C. W. R. Wade, et al. “Biocompatibility of eight poly(organophosphazenes),” in Organomet. Polym., C. E. Carraher, J. E. Sheats and C. U. Pitman, Jr., Eds., Academic Press, New York, 1978, pp. 283-288; and H. R. Allcock and T. J. Fuller, “Synthesis and Hydrolysis of Hexakis(imidazolyl)cyclotriphosphazene,” J. Am. Chem. Soc., 103:2250-2256 (1981). The inclusion of these hydrolytic side chains will affect the degradation of the polymer as an implantable and biodegradable material as well as vary the support of osteogenic cells for bone and tissue implants, as shown by Laurencin, et al., “Use of polyphosphazenes for skeletal tissue regeneration,” J. Biomats. Res., 27:963-973 (1993).

PLA, PGA and PLA/PGA copolymers are particularly useful for forming the biocompatible and biodegradable matrices employed in the present invention. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. No.2,683,136; 3,531,561 to Trehu. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. PGA polymers and their properties are described in more detail in “Cyanamid Research Develops World's First Synthetic Absorbable Suture,” Chemistry and Industry, 905 (1970).

The rate of erosion of the matrix is directly related to the molecular weights of PLA, PGA or PLA/PGA. The higher molecular weights, weight average molecular weights of 90,000 or higher, result in polymer matrices which retain their structural integrity for longer periods of time; while lower molecular weights, weight average molecular weights of 30,000 or less, result in both slower release and shorter matrix lives. Poly(lactide-co-glycolide) (50:50), degrades in about six weeks following implantation.

When cells are used in the biocompatible material, one or more polymers used in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC), and bond structure by infrared (IR) spectroscopy, with respect to toxicology by initial screening tests involving Ames assays and in vitro teratogenicity assays, and implantation studies in animals for immunogenicity, inflammation, release and degradation studies.

In another embodiment, one or more of the polymers is used form a fibrous or sponge type matrix (analogous to the sponge like matrix in cancellous bone) for use as a carrier for the runx2 protein or a nucleic acid or a vector encoding the runx2 protein. In an alternate embodiment, sterile and antigen-free cancellous bone itself is used a primary or secondary carrier for the runx2 protein or a nucleic acid or a vector encoding the runx2 protein.

In yet another embodiment, polymers that form hydrogels are used as carriers for cells that are transformed to express Runx2 that are suspended or seeded thereon. In another embodiment, the polymers that form hydrogels are used as carriers for the Runx2 protein or a nucleic acid or an expression vector encoding the Runx2 protein. Examples of such hydrogel forming polymers are the crosslinked acrylates or methacrylates, such as disclosed in U.S. Pat. No. 6,552,103, which issued to Bertozzi, et al. on Apr. 22, 2003, entitled “Biomimetic hydrogel materials;” U.S. Pat. No. 6,379,690, which issued to Blanchard, et al. on Apr. 30, 2002, entitled “Keratin-based hydrogel for biomedical applications and method of production;” U.S. Pat. No. 6,368,356, which issued to Zhong, et al. on Apr. 9, 2002, entitled “Medical devices comprising hydrogel polymers having improved mechanical properties;” U.S. Pat. No. 6,361,797, which issued to Kuzma, et al. on Mar. 26, 2002, entitled “Hydrogel compositions useful for the sustained release of macromolecules and methods of making same;” and U.S. Pat. No. 6,333,194, which issued to Levy, et al. on Dec. 25, 2001, entitled “Hydrogel compositions for controlled delivery of virus vectors and methods of use thereof,” all of which are expressly incorporated herein in their entirety by reference thereto.

In a preferred embodiment, which is not meant to be limiting, the pharmaceutically acceptable carrier is formed from either natural or synthetic polymers, or combination thereof. Runx2, as a protein [SEQ ID NO 2, or SEQ ID NO: 4], is loaded into the polymeric carrier or physically entrapped within the polymeric carrier during the polymerization process.

When the active agent is a nucleotide sequence [SEQ ID NO: 1 or 3] encoding Runx2, the nucleotide sequence is operatively connected to one or more promoters and or enhancers for obtaining nucleotide expression. Methods for operatively adding promoters and enhancers to a polynucleotide to obtain polynucleotide expression are well known in the art, as disclosed in U.S. Pat. No. 6,241,982, which issued to Barber, et al. on Jun. 5, 2001 and is entitled “Method for treating brain cancer with a conditionally lethal gene;” and in U.S. Pat. No. 6,221,646, which issued to Dwarki, et al. on Apr. 24, 2001 and is entitled “Materials and methods for simplified AAV production,” both of which are incorporated by reference herein in their entirety. The resulting expressible polynucleotide is loaded into the delivery system comprised of a natural polymer, a synthetic polymer, or combination thereof, or physically entrapped within the delivery system during the polymerization process. Preferably, the nucleotide system is in the form of a plasmid or a vector.

In a further embodiment of the pharmaceutical composition, device and method of the present invention, cells that are genetically altered to express Runx2 are seeded onto and into the delivery system comprised of a natural polymer, a synthetic polymer, or combination thereof, for implantation to repair a bone defect. In one embodiment, the genetically altered cells are seeded on the matrix in vivo, or in another embodiment, the genetically altered cells are mixed into the matrix in vitro prior to polymerization and implantation, or in another embodiment, the matrix is implanted immediately upon seeding with the genetically altered cells.

Collagen

Collagens are natural biocompatible and biodegradable polymeric molecules that are suitable carriers for the active Runx2 agents of the present invention. Collagen is typically derived from bone (whose mineral component has been removed, such as with 0.5N HCl), or skin or tendon. Demineralized bone matrix (DBM) was the initial carrier of choice for implantation of purified or recombinant BMPs. DBM is comprised of mostly insoluble, highly crosslinked type I collagen, although other non-characterized collagens and non-collagenous proteoglycans may be present. Preferably, non-collagenous proteoglycans and other potentially antigenic proteins are removed from the DBM, such as by using the process disclosed in commonly assigned U.S. Pat. No. 6,482,584, which issued to “Mills, et al. on Nov. 19, 2002, entitled “Cyclic implant perfusion, cleaning and passivation process,” and which is incorporated herein in its entirety by reference thereto.

Another suitable naturally occurring polymer that is principally collagen is guanidine/urea extracted DBM (from which soluble bone morphogenetic activity is removed), preferably subjected to the above referenced perfusion, cleaning and passivation process. Such DBM is an excellent osteoconductive template by itself in bridging small bony defects. Since DBM is a bone-derived scaffold, it can provide an ideal template for the attachment, and differentiation of osteoprogenitor cells. When implanted with BMP, DBM can potentially affect the osteoinductive activity, thus participating directly in the mineralization process. In a clinical setting, however, allogeneic DBM may elicit an inflammatory or immunogenetic response. Other potential concerns with DBM include the feasibility of disease transmission with allografts and, as a particulate carrier, it is critical for DBM particles to be retained at the implant site to prevent bone formation at undesirable sites.

Limitations of DBM have inspired the exploration of reconstituted collagens from other sources as alternative BMP carriers. Different types of collagens have been purified on a large scale. The purification process typically yields a collagen solution or dispersion, which can be fabricated into macroporous scaffolds in an implantable format. Macroporosity is important for cellular access, since BMP action is based on the cell invasion of a carrier, hence the need for a freely accessible space within a carrier. The manufacturing process also may allow control of over the wettability, mechanical properties and geometry (pore size and connectivity) of the carriers. Reduction of collagen immunogenicity has been observed following enzymatic treatment, e.g. by pepsin, to remove the collagen telopetide fraction. In addition, both physical (e.g., dehydrothermal, UV-irradiation) and chemical crosslinking strategies (e.g., aldehydes, carbodiimides, etc.) have been explored to minimize immunogenicity and to provide control over in vivo biodegradation. It is noteworthy that the in vivo cellular reaction and biodegradation of collagen scaffolds are different when they are implanted with BMP as opposed to implanted alone.

In a preferred embodiment, Runx2, as a protein, or as an expressible polynucleotide encoding Runx2, or as a cell transformed to express Runx2 is incorporated into the delivery system comprised of collagen or DBM for repair of the bone defect. The cells may be cultured on the carrier matrix in vitro prior to implantation, or seeded onto the matrix immediately prior to implantation.

Other Matrix Materials

Another class of materials for making the carrier matrix is hydroxyapatite, or a similar ceramic formed of tricalcium phosphate (TCP) or calcium phosphate (CaPO₄). Calcium hydroxyapatites occur naturally as geological deposits and in normal biological tissues, principally bone, cartilage, enamel, dentin, and cementum of vertebrates and in many sites of pathological calcifications such as blood vessels and skin. Synthetic calcium hydroxyapatite is formed in the laboratory either as pure Ca₁₀(PO₄)₆(OH)₂ or hydroxyapatite that is impure, containing other ions such as carbonate, fluoride, chloride for example, or crystals deficient in calcium or crystals in which calcium is partly or completely replaced by other ions such as barium, strontium and lead. Essentially none of the geological and biological apatites are “pure” hydroxyapatite since they contain a variety of other ions and cations and may have different ratios of calcium to phosphorous than the pure synthetic apatites.

In general, the crystals of pure synthetic apatites, geological apatites and many impure synthetically produced apatites are larger and more crystalline than the biological crystals of bone, dentin, cementum and cartilage. The crystals of bone, dentin and cementum are very small, irregularly shaped, very thin plates whose rough average dimensions are approximately 10 to 50 angstroms in thickness, 30 to 150 angstroms in width, and 200 to 600 angstroms in length. The synthetic materials are highly diverse, as reported in the literature. For example, the characterization of four commercial apatites was reported by Pinholt, et al., J. Oral Maxillofac. Surg. 50(8), 859-867 (August 1992); J. Cariofac. Surg. 1(3), 154-160 (July 1990) reports on a protein, biodegradable material; Pinholt, et al., Scand. J. Dent. Res. 99(2), 154-161 (April 1991) reports on the use of a bovine bone material called Bio-Oss™; Friedman, et al., Arch. Otolaryngol. Head Neck Surg. 117(4), 386-389 (April 1991) and Costantino, et al., Arch. Otolaryngol. Head Neck Surg. 117(4), 379-384 (April 1991) report on a hydroxyapatite cement; Roesgen, Unfallchirurgle 16(5), 258-265 (October 1990), reports on the use of calcium phosphate ceramics in combination with autogenic bone; Ono, et al., Biomaterials 11(4), 265-271 (May 1990) reports on the use of apatite-wollastonite containing glass ceramic granules, hydroxyapatite granules, and alumina granules; Passuti, et al., Clin. Orthop. 248, 169-176 (November 1989) reports on macroporous calcium phosphate ceramic performance; Harada, Shikwa-Gakuho 89(2), 263-297 (1989) reports on the use of a mixture of hydroxyapatite particles and tricalcium phosphate powder for bone implantation; Ohgushi, et al., Acta Orthop. Scand. 60(3), 334-339 (1989) reports on the use of porous calcium phosphate ceramics alone and in combination with bone marrow cells; Pochon, et al., Z-Kinderchir. 41(3), 171-173 (1986) reports on the use of beta-tricalcium phosphate for implantation; and Glowacki, et al., Clin. Plast. Surg. 12(2), 233-241 (1985), reports on the use of demineralized bone implants.

As used herein, all of these materials are generally referred to as “hydroxyapatite”. In the preferred form, the hydroxyapatite is particles having a diameter between approximately ten and 100 microns in diameter, most preferably about 50 μm in diameter.

Calcium phosphate ceramics can be used as implants in the repair of bone defects because these materials are non-toxic, non-immunogenic, and are composed of calcium and phosphate ions, the main constituents of bone (Jarcho, 1981; Frame, J. W., “Hydroxyapatite as a biomaterial for alveolar ridge augmentation,” Int. J. Oral Maxillofacial Surgery, 16, 642-55 (1987); Parsons, et al. “Osteoconductive Composite Grouts for Orthopedic Use,” Annals N.Y. Academy of Sciences, 523, 190-207 (1988)). Both tricalcium phosphate (TCP) [Ca₃(PO₄)₂] and hydroxyapatite (HA) [Ca₁₀(PO₄)₆(OH)₂] have been widely used. Calcium phosphate implants are osteoconductive, and have the apparent ability to become directly bonded to bone. As a result, a strong bone-implant interface is created.

Calcium phosphate ceramics have a degree of bioresorbability, which is governed by their chemistry and material structure. High density HA and TCP implants exhibit little resorption, while porous ones are more easily broken down by dissolution in body fluids and resorbed by phagocytosis. However, TCP degrades more quickly than HA structures of the same porosity in vitro. HA is relatively insoluble in aqueous environments. However, the mechanical properties of calcium phosphate ceramics make them ill suited to serve as a structural element under load bearing circumstances. Ceramics are not preferred since they are brittle and have low resistance to impact loading.

In another embodiment, the pharmaceutically acceptable carrier utilized in the composition, device or method of the present invention is comprised of hydroxyapatite or a similar calcium phosphate ceramic. Runx2, as a protein, is incorporated into the delivery system to be used in the repair of a bone defect. In a second preferred embodiment, Runx2, as a gene, is combined with the delivery system comprised of hydroxyapatite or a similar calcium phosphate ceramic. In yet another embodiment, cells genetically engineered to express Runx2 are seeded onto and into the carrier system comprised of hydroxyapatite or a similar calcium phosphate ceramic. In this latter embodiment, the cells are cultured on the carrier in vitro prior to implantation, or are seeded onto the carrier prior to implantation.

Bioglasses

Other pharmaceutically acceptable carriers suitable for use in the composition, device and method of the present invention include the bioglasses. Bioglassses are silico-phosphatic chains that bond ionically to compounds such as CaO, CaF₂, P₂O₅, Na₂O, K₂O, ZnO, Al₂O₃, TiO₂, CuO, and NiO. Furthermore, bioglasses undergo ionic translocations; consequently, biomedical devices composed of bioglass can exchange ions or molecular groups with the contiguous physiological milieu (an osseous recipient site). This property enables bioglass devices to osseointegrate, that is, to chemically bond to bone. Bioresorbable, synthetically derived bioglasses have been developed and approved by the FDA for repairing bony defects in alveolar bone (Biogran, Orthovita, Malvern, Pa.). These biocompatible, bioresorbable, synthetically derived bioglasses are also useful as a pharmaceutically acceptable carrier in the pharmaceutical composition, device and method of the present invention.

In one embodiment, the protein form of Runx2 is intermixed with the delivery system comprised of biocompatible, bioresorbable, synthetically derived bioglass. In another embodiment, the genetic form of Runx2 is integrated within the delivery system comprised of biocompatible, bioresorbable, synthetically derived bioglass. In a further embodiment, cells genetically engineered to express Runx2 are seeded onto and into the delivery system comprised of biocompatible, bioresorbable, synthetically derived bioglass. In this latter embodiment, the cells are cultured on the carrier in vitro prior to implantation, or are seeded onto the carrier prior to implantation.

Matrix Configuration of the Carrier

For an organ to be constructed, successfully implanted, and function, the matrices must have sufficient surface area and exposure to nutrients such that cellular growth and differentiation can occur prior to the ingrowth of blood vessels following implantation. The time required for successful implantation and growth of the cells within the matrix is greatly reduced if the area into which the matrix is implanted is prevascularized. After implantation, the configuration must allow for diffusion of nutrients and waste products and for continued blood vessel ingrowth as cell proliferation occurs.

The organization of the tissue may be regulated by the microstructure of the matrix. Specific pore sizes and structures may be utilized to control the pattern and extent of fibrovascular tissue ingrowth from the host, as well as the organization of the implanted cells. The surface geometry and chemistry of the matrix may be regulated to control the adhesion, organization, and function of implanted cells or host cells.

In the preferred embodiment, the pharmaceutically acceptable carrier is a matrix formed of polymers having a fibrous structure, which has sufficient interstitial spacing typically in the range of 100 to 300 microns. A microporous structure (pores 205 μm in diameter) has been shown to be too small to permit the ingrowth of cells, as reported by Friedlander, G. E. and V. M. Goldberg, Bone and Cartilage Allografts, Park Ridge: American Academy of Orthopedic Surgeons, 1991; Jarcho, M. “Calcium Phosphate Ceramics as Hard Tissue Prosthetics,” Clinical Orthopedics and Related Research, 157, 259-78 (1981). As used herein, “fibrous” includes one or more fibers that are entwined with itself, multiple fibers in a woven or non-woven mesh, and sponge like devices.

The overall, or external, matrix configuration is dependent on the tissue which is to reconstructed or augmented. These are readily determined by the surgeon based on the defect to be corrected.

Polymers for Forming Hydrogels

Another pharmaceutically acceptable carrier for use in the pharmaceutical composition, device and method of the present invention is a biocompatible and biodegradable polymer which forms a hydrogel. Polymers that form ionic hydrogels, which are malleable, are suitable carriers for Runx2, either in protein, nucleic acid or transformed cellular form. Furthermore, injecting a suspension of cells in a polymer solution improves the reproducibility of cell seeding throughout a device, and protects the cells from shear forces or pressure induced necrosis, and aids in defining the spatial location of cell delivery. The injectable polymer is also utilized to deliver cells and promote the formation of new tissue without the use of any other matrix. In a preferred embodiment, the hydrogel is produced by cross-linking the ionic salt of a polymer with ions, whose strength increases with either increasing concentrations of ions or polymer. The polymer solution is mixed with the cells to be implanted to form a suspension, which is then injected directly into a patient prior to hardening of the suspension. The suspension subsequently hardens over a short period of time due to the presence in vivo of physiological concentrations of ions such as calcium in the case where the polymer is a polysaccharide such as alginate.

A hydrogel is defined as a substance formed when an organic polymer (natural or synthetic) is cross-linked via covalent, ionic, or hydrogen bonds to create a three-dimensional open-lattice structure, which entraps water molecules to form a gel. Examples of materials which can be used to form a hydrogel include polysaccharides such as alginate, polyphosphazenes, and polyacrylates such as hydroxyethyl methacrylate (HEMA), which are crosslinked ionically, or block copolymers such as Pluronics™ or Tetronics™, polyethylene oxide-polypropylene glycol block copolymers which are crosslinked by temperature or pH, respectively. Other suitable materials include natural polymers such as fibrin or collagen, or synthetic polymers such as polyvinylpyrrolidone or polymers of hyaluronic acid.

In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Alginate can be ionically cross-linked with divalent cations, in water, at room temperature, to form a hydrogel matrix. Due to these mild conditions, alginate has been the most commonly used polymer for hybridoma cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol and glucosyl.

The water soluble polymer with charged side groups is crosslinked by reacting the polymer with an aqueous solution containing multivalent ions of the opposite charge, either multivalent cations if the polymer has acidic side groups or multivalent anions if the polymer has basic side groups. The preferred cations for cross-linking of the polymers with acidic side groups to form a hydrogel are divalent and trivalent cations such as copper, calcium, aluminum, magnesium, strontium, barium, and tin, although di-, tri- or tetra-functional organic cations such as alkylammonium salts, e.g., R₃N⁺-+NR₃ can also be used. Aqueous solutions of the salts of these cations are added to the polymers to form soft, highly swollen hydrogels and membranes. The higher the concentration of cation, or the higher the valence, the greater the degree of cross-linking of the polymer. Concentrations from as low as 0.005M have been demonstrated to cross-link the polymer. Higher concentrations are limited by the solubility of the salt. The preferred anions for cross-linking of the polymers to form a hydrogel are divalent and trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethyleneimine and polylysine. These are commercially available. One polycation is poly(L-lysine), examples of synthetic polyamines are: polyethyleneimine, poly(vinylamine), and poly(allyl amine). There are also natural polycations such as the polysaccharide, chitosan. Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO₃H groups such as sulfonated polystyrene, and polystyrene with carboxylic acid groups.

In a preferred embodiment, which is not meant to be limiting, the pharmaceutically acceptable carrier comprises a hydrogel. The Runx2, as a protein, is incorporated into the hydrogel or physically entrapped within the hydrogel during the polymerization process.

In another embodiment, a polynucleotide encoding Runx2 is integrated into the delivery system comprised of a hydrogel, or physically entrapped within the hydrogel during the polymerization process.

In a further embodiment, cells genetically altered to express Runx2 are seeded onto or dispersed into the hydrogel for implantation to repair the bone defect. The cells are cultured on the matrix in vitro prior to implantation, or are seeded onto the hydrogel prior to implantation

Runx2 Delivery

Runx2 must be “delivered” to the patient in the proper carrier. Depending upon the form of Runx2 being provided, the carrier (i.e., delivery system) ensures that the Runx2 is localized, protected from lytic enzymes, phagocytotic cells, and sustained at the delivered site for the appropriate spatial and temporal cellular interactions. These fundamental criteria for the pharmaceutical composition, device and method of the present invention are consistent with a physiologically effective bone tissue engineered therapy. As already noted above, the three modes for delivery of Runx2 include, but are not limited to, protein delivery, polynucleotide delivery, and delivery of a genetically engineered cell (typically, a plurality of genetically engineered cells expressing Runx2).

Protein Delivery

In a preferred embodiment of the present invention, Runx2 [SEQ ID NO: 2 or 4] or carboxy truncated fragments thereof, are combined and dispersed in a suitable carrier preparation using any of the methods described herein. In general, a therapeutically effective amount of Runx2 is combined with a pharmaceutically acceptable suitable carrier. The optimal concentration of Runx2 for a specific combination and tissue type may be determined empirically by those of skill in the art according to the procedures set forth herein.

Liposomes

Additional suitable carriers for Runx2, either in protein form or as its encoding polynucleotide, include microspheres, liposomes, other microparticulate delivery systems or sustained release formulations in conjunction with a suitable matrix material placed in, near, or otherwise in communication with a bone defect or the bloodstream bathing the bone defect.

Liposomes containing a Runx2 of this invention are prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily, the liposomes are of the small (about 200-800 Angstroms) unilamellar type in which the lipid content is greater than about 30 mol. % cholesterol. The proportion of cholesterol is selected to control the optimal rate of Runx2 release.

Runx2 may also be attached to liposomes containing other biologically active molecules such as immunosuppressive agents, cytokines, etc., to modulate the rate and characteristics of tissue induction. Attachment of Runx2 to liposomes may be accomplished by any known cross-linking agent such as heterobifunctional cross-linking agents that have been widely used to couple toxins or chemotherapeutic agents to antibodies for targeted delivery. Conjugation to liposomes is also accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

Gene Delivery

In a preferred embodiment, tissue engineered therapy includes a DNA plasmid vector for Runx2 non-covalently associated with a demineralized bone matrix product (DBM) administered to an osseous defect. However, additional nucleic acid delivery systems include demineralized bone matrix (DBM) preparations, injectable calcium phosphate cements, calcium sulfates, tricalcium phosphates, hyaluronic acid, gelatin, alginate, fibrin glue products, collagens, collagen-calcium-phosphate combinations, polymers (e.g., poly (lactide-co-glycolides), polylactides, polyglycolides, polyanhydrides, polyphosphazenes, polycarbonates, polyurethane) and all such modifications).

Genetic Engineering of Cells

Delivering the genetic material into a targeted host cell is accomplished by several techniques. Targeting vectors have been successfully applied for use either ex vivo or in situ and re-implanted. Ex vivo, an osteoblast or osteogenic precursor cell population can be altered genetically to express a preferred molecule, e.g. Runx2 Thereafter, the cells could be selected, expanded in number, and transplanted to a patient. Alternately, in situ delivery involves the direct genetic manipulation of a host cell, either targeted or general. Targeted gene therapy is a rational and efficient alternative to methods that use exogenous growth factors. Cells

The cells that can be manipulated for use in the methods described herein include almost any type of tissue. These are typically normal mammalian cells, preferably of the same species as the ultimate recipient, most preferably of the same origin as the recipient, although the method can be practiced using xenotransplants which have been altered to decrease the likelihood of rejection, for example, by expression of a complement inhibitor such as CD59, or masking of sugar residues. These techniques are known to those skilled in the art, and have been commercially developed using pigs as the donors, by Alexion Pharmaceuticals, Conn., and DNX, PA.

Cell type will typically be selected based on the tissue to be repaired or formed. For example, chondrocytes or fibroblasts can be selected to form cartilage; muscle cells to form muscle. Undifferentiated, or less differentiated, cells may be preferred in some situation. Representative of these cell types include stem cells and mesenchymal cells. In a preferred embodiment, mesenchymal cells are obtained from periosteum, then genetically engineered. Specific examples of cells that are suitable for aiding healing, repair or formation of bone include osteocytes/osteoblasts and periosteal cells, particularly in combination with BMP-2-15 and/or IGF. Specific examples of cells that aid healing, repair or formation of cartilage include chondrocytes and periosteal cells, particularly in combination with CGF and/or TGF-beta. Specific examples of cells that aid healing, repair or formation of skin include dermal and epidermal cells, particularly in combination with PDGF, VEGF, IGF, and GH. Specific examples of cells that aid healing, repair or formation of nervous tissue include nerve cells and support cells, particularly in combination with NGF. In most cases, cells will be obtained from a tissue biopsy, which is digested with collagenase or trypsin to dissociate the cells. Alternatively, cells are obtained from established cell lines or from embryonic cell sources.

The cells can be transfected using any appropriate means, including viral vectors, as shown by the example, chemical transfectants, or physico-mechanical methods such as electroporation and direct diffusion of DNA. See, for example, Wolff, Jon Aal, et al, “Direct gene transfer into mouse muscle in vivo,” Science, 247: 1465-1468 (1990); and Wolff, Jon A, “Human dystrophin expression in mdx mice after intramuscular injection of DNA constructs,” Nature, 352:815-818 (1991). Plasmid DNA, which function episomally, has been with liposome encapsulation, CaPO₄ precipitation and electroporation as an alternative to viral transfections. Clinical trials with liposome encapsulated DNA in treating melanoma is reported by Nabel, J. G., et al., “Direct gene transfer with DNA-liposome complexes in melanoma: Expression, biological activity and lack of toxicity in humans,” Proc. Nat. Acad. Sci. U.S.A., 90:11307-11311 (1993); Felgner, Philip L, “Lipofectamine reagent: A new, higher efficiency polycationic liposome transfection reagent,” Focus/Gibco, 15, 73-78, 1993; Partridge, Terence A, “Muscle transfection made easy, “Nature, 352, 757-758, 1991; Wilson, James M, “Vehicles for gene therapy,” Nature, 365, 691-692, 1993; Wivel, et al., “Germ-line gene modification and disease prevention: Some medical and ethical perspectives,” Science, 262, 533-538, 1993; and Woo, Savio L Cal, et, “In vivo gene therapy of hemophilia B: sustained partial correction in Factor IX-deficient dogs,” Science, 262, 117-119, 1993.

As used herein, vectors are agents that transport the gene into the cell without degradation and include a promoter yielding expression of the gene in the cells into which it is delivered. Promoters can be general promoters, yielding expression in a variety of mammalian cells, or cell specific, or even nuclear versus cytoplasmic specific. These are known to those skilled in the art and can be constructed using standard molecular biology protocols. Vectors have been divided into two classes:

a) Biological agents derived from viral, bacterial or other sources.

b) Chemical/physical methods that increase the potential for gene uptake, directly introduce the gene into the nucleus or target the gene to a cell receptor.

Biological Vectors

Viral vectors have higher transaction (ability to introduce genes) abilities than do most chemical or physical methods to introduce genes into cells.

Retroviral vectors are the vectors most commonly used in clinical trials, since they carry a larger genetic payload than other viral vectors. Retroviral vectors are RNA vectors that stably integrate into the host genome. However, they are not capable of infecting non-proliferating cells. Suitable retroviral vectors are human based retroviruses or ampohotropic mammalian viral vectors, such as the Moloney murine leukemia virus. Examples of suitable retroviral vectors are disclosed in U.S. Pat. No. 6,071,512, which issued to Kriegler, et al. on Jun. 6, 2000 and is entitled “Infective protein delivery system;” U.S. Pat. No. 6,241,982, which issued to Barber, et al. on Jun. 5, 2001 and is entitled “Method for treating brain cancer with a conditionally lethal gene;” and U.S. Pat. No. 5,888,502, which issued to Guber, et al. on Mar. 30, 1999 and is entitled “Recombinant retroviruses;” all of which are hereby incorporated by reference herein in their entirety.

Adenovirus vectors are relatively stable and easy to work with, have high titers, and can be delivered in aerosol formulation. The use of adenoviral vectors is well known in the art, such as disclosed in U.S. Pat. No. 5,882,877, which issued to Gregory, et.al. on Mar. 16, 1999 and is entitled “Adenoviral vectors for gene therapy containing deletions in the adenoviral genome,” which is incorporated by reference herein in its entirety.

Pox viral vectors are large and have several sites for inserting genes, they are thermostable and can be stored at room temperature.

Plasmids are not integrated into the genome and the vast majority of them are present only from a few weeks to several months, so they are typically very safe. However, they have lower expression levels than retroviruses and since cells have the ability to identify and eventually shut down foreign gene expression, the continuous release of DNA from the polymer to the target cells substantially increases the duration of functional expression while maintaining the benefit of the safety associated with non-viral transfections. The use of plasmids as expression vectors is well known in the art, such as disclosed in U.S. Pat. No. 5,981,501, which issued to Wheeler, et al. on Nov. 9, 1999 and is entitled “Methods for encapsulating plasmids in lipid bilayers,” which is incorporated by reference herein in its entirety.

Chemical/Physical Vectors

Other methods to directly introduce genes into cells or exploit receptors on the surface of cells include the use of liposomes and lipids, ligands for specific cell surface receptors, cell receptors, and calcium phosphate and other chemical mediators, microinjections directly to single cells, electroporation and homologous recombination. Liposomes are commercially available from Gibco BRL, for example, as LIPOFECTION® and LIPOFECTACE®, which are formed of cationic lipids such as N-[1-(2,3 dioleyloxy)-propyl]-n,n,n-trimethylammonium chloride (DOTMA) and dimethyl dioctadecylammonium bromide (DDAB). Numerous methods are also published for making liposomes, known to those skilled in the art.

Seeding and Implantation of the Matrix

Cells can be implanted directly into a defect in an amount effective to promote repair. Alternatively, the cells are seeded onto and into a matrix for implantation to repair the defect. The cells may be cultured on the matrix in vitro prior to implantation, or implanted immediately upon seeding.

In the preferred embodiment, the cells are seeded onto and into the matrix, then cultured for a time dependent on the matrix material which is employed. For example, when biodegradable polymeric matrices are used, cells will typically be cultured for between one and five weeks. Cells cultured on matrices formed of a material such as hydroxyapatite can be cultured for between one and ten weeks. The matrix is then surgically implanted at the site of the defect to be corrected, using standard surgical techniques.

In the case of a hydrogel, the polymer is dissolved in an aqueous solution, preferably a 0.1 M potassium phosphate solution, at physiological pH, to a concentration forming a polymeric hydrogel, for example, for alginate, of between 0.5 to 2% by weight, preferably 1% alginate. The isolated cells are suspended in the polymer solution to a concentration of between 1 and 50 million cells/ml, most preferably between 10 and 20 million cells/ml. This is then injected into the site of the bone defect where the repair is needed.

Cells are tested for expression of the transfected gene prior to or days to weeks later following implantation. Expression will typically be required to continue for one to two months to effect healing of a bone defect.

Cell Assays

Cells are examined and characterized by both phase contrast microscopy and light reflectance microscopy. Determination of osteoblast-like character is performed through a variety of methods. Osteocalcin production is measured using the method of Gundberg, Lian, and Gallop, Clin. Chem. Acta 128, 1 (1983). Monolayer cultures are washed and incubated in serum-free medium containing 10 mg/cc bovine serum albumin in the absence or presence of 10 nM 1,25(dihydroxy)vitamin D₃ for 48 hours. The medium is analyzed by radioimmunoassay for osteocalcin at the end of the incubation period.

A number of assays can be used to evaluate the cells. For example, cAMP response to parathyroid hormone (PTH) is measured by incubated cultures near confluency with 1 mM isobutylmethylxanthine alone or in combination with 0.2 to 200 ng/cc of PTH with 1-10 ng/cc calcitonin for 10 minutes at 37° C. Incubations are halted by quickly transferring the cultures to ice, washing rapidly with ice-cold Tyrode's solution, and then adding ice-cold ethanol containing 2 mM HCL. After homogenizing the cells in ethanol for 5 seconds, the samples are dried at 100° C. and cAMP concentrations are measured by radioimmunoassay.

Alkaline phosphatase activity of the cell lysates is determined in a similar manner using the method of Luben, Wong and Cohn, Endocrinology 35, 778 (1983) with n-nitro-phenylphosphate as substrate. Collagen synthesis is analyzed according to Schwartz. Cultures are incubated for 48 hours in Minimal Essential Medium (Gibco) containing fetal bovine serum (10%), sodium ascorbate (50 microgram/ml), B-aminopropionitrile and tritiated proline (50 UCi/cc). Collagen from the culture medium and cell layer is isolated, treated with pepsin, and after the addition of carrier type I collagen, analyzed by SDS-PAGE under reducing and non-reducing conditions, as described by Neville, J. Biol. Chem. 246, 6326 (1971). Bands of carrier protein are identified by staining with Coomassie blue. The gels are then sliced into 25 equal segments each of which is monitored for radioactive content. Collagen distribution is calculated according to the method of Goldberg, et al., Biochem. Pharmacol. 29, 869 (1980).

Other Bioactive Molecules

The biocompatible, biodegradable carrier used in the composition, device and method of the present invention may also incorporate other bioactive molecules that enhance the rate of bone repair. Other polynucleotides may be added which encode a useful protein, for example, a specific growth factor, morphogenesis factor, structural protein, or cytokine which enhances the temporal sequence of wound repair, alters the rate of proliferation, increases the metabolic synthesis of extracellular matrix proteins, or directs phenotypic expression in endogenous cell populations. Representative genes encoding proteins include bone growth factor genes (BMPs, IGF) for bone healing, cartilage growth factor genes (CGF, TGF-beta) for cartilage healing, nerve growth factor genes (NGF) for nerve healing, and general growth factors important in wound healing, such as platelet-derived growth factor (PDGF), vascular endothelial growth factor (VEGF), insulin-like growth factor (IGF-1), keratinocyte growth factor (KGF), endothelial derived growth supplement (EDGF), epidermal growth factor (EGF), basic fibroblast growth factor (FGF) for wound and skin healing.

In addition, members of the TGF-beta superfamily appear to play a central role in mesenchymal differentiation, including cartilage and bone formation. TGF-beta enhances bone cell proliferation. The TGF-beta superfamily includes the bone morphogenic proteins, including BMP-2-5 and Insulin-like growth factor (IGF). These can be further divided into three distinct subfamilies: BMP-2, BMP-3, and BMP-7. The different isoforms have different activities in bone morphogenesis and repair. They are closely related to factors that are involved in a variety of developmental processes during embryogenesis. For example, any of BMP 2-7 can be used to induce bone formation and differentiation. IGF has been shown to increase bone formation, promoting fracture healing and inducing bone growth around implants, in conjunction with TGF-beta and BMPs. Other osteoinductive factors such as osteogenin (BMP-3), a skeletal growth factor (SGF), and osteoblast-derived (BDGFs) have also been recently discovered. Other factors shown to act on cells forming bone, cartilage or other connective tissue include retinoids, fibroblast growth factors (FGFs), growth hormone (GH), and transferrin. Proteins specific for cartilage repair include cartilage growth factor (CGF) and TGF-beta. The local microenvironment also affects differentiation and development of cells.

Preferred examples for bone repair and/or treatment of osteoporosis uses periosteal or other mesenchymal stem cells or osteocytes/osteoblasts transfected with bone growth factor genes such as bone morphogenetic protein (BMP) family genes, including BMP 2-15; for cartilage repair uses periosteal cells or chondrocytes trarisfected with cartilage growth factor genes such as transforming growth factor-.beta. (TGF-.beta.) and cartilage growth factor (CGF); for wound healing uses dermal or epidermal cells transfected with growth factor genes such as platelet derived growth factor (PDGF), epidermal growth factor (EGF), vascular endothelial growth factor (VEGF), keratinocyte growth factor (KGF), fibroblast growth factor (FGF), endothelial derived growth supplement (EDGS), or insulin-like growth factor (IGF); for nerve repair (central and/or peripheral) uses neural cells and neural support cells transfected with nerve growth factor (NGF) gene.

In a preferred embodiment, a biologically active molecule, which enhances the rate of bone repair, either in a protein or a genetic form, is combined with a delivery system and a form of Runx2 for implantation to repair a bone defect.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A pharmaceutical composition comprising in combination a Runx2 protein, a polynucleotide encoding the Runx2 protein, or a cell that has been transformed with a polynucleotide encoding the Runx2 protein, in a pharmaceutically acceptable carrier, said carrier comprising a biocompatible, biodegradable polymeric matrix.

2. The pharmaceutical composition of claim 1, wherein the biocompatible, biodegradable polymeric matrix decomposes over 1-3 months.

3. The pharmaceutical composition of claim 2, wherein the biocompatible, biodegradable polymeric matrix decomposes over 2-3 months.

4. The pharmaceutical composition of claim 2, wherein the biocompatible, biodegradable polymeric matrix decomposes over 1-3 months and absorbed by the body so as to temporarily release the Runx2 protein locally in vivo at the site where bone repair and/or osteoblastic activity is needed.

5. The pharmaceutical composition of claim 1, wherein the biocompatible, biodegradable polymeric matrix is a hydrogel.

6. The pharmaceutical composition of claim 1, wherein the biocompatible, biodegradable polymeric matrix is a hydrated collagen.

7. A medical device comprising in combination:
a sterile and substantially antigen-free, pre-shaped allograft or xenograft bone implant;
a Runx2 protein, a polynucleotide encoding the Runx2 protein, or a cell that has been transformed with a polynucleotide encoding the Runx2 protein;
in a pharmaceutically acceptable carrier comprising a biocompatible, biodegradable polymeric matrix.

8. The medical device of claim 7, wherein the bone implant is an allograft bone implant.

9. The medical device of claim 7, wherein the bone implant has one or more cavities formed therein suitable for receiving the Runx2 protein, the polynucleotide encoding the Runx2 protein, or a cell that has been transformed with a polynucleotide encoding Runx2 protein, in said pharmaceutically acceptable carrier.

10. The medical device of claim 7, wherein the bone implant is partially demineralized.

11. The medical device of claim 8, wherein the allograft bone implant is a composite device composed of three or more pieces of sterile and substantially antigen-free, pre-shaped allograft bone.

12. The medical device of claim 11, wherein one of the pieces of sterile and substantially antigen-free, pre-shaped allograft bone is a piece of cancellous bone.

13. A method for repairing a bone defect comprising administering to a mammalian patient at the site in need of treatment a pharmaceutical composition, comprising in combination the Runx2 protein, a polynucleotide encoding the Runx2 protein, or a cell that has been transformed with a polynucleotide encoding Runx2 protein, in a pharmaceutically acceptable carrier wherein the carrier is a bio-compatible, biodegradable polymeric matrix.

14. The method of claim 13, wherein biocompatible, biodegradable polymeric matrix is slowly decomposed and absorbed by the body so as to temporarily release the Runx2 protein locally in vivo at the site where bone repair and/or osteoblastic activity is needed.

15. The method of claim 14, wherein biocompatible, biodegradable polymeric matrix decomposes in the mammalian patient within 1 to 3 months of being administered.

16. The method of claim 15, wherein biocompatible, biodegradable polymeric matrix decomposes in the mammalian patient within 2 to 3 months of being administered.

17. The method of claim 13, further comprising administering to a mammalian patient at the site in need of treatment a sterile and substantially antigen-free, pre-shaped allograft bone implant sized and shaped to fit the bone defect.

18. The method of claim 13, wherein the Runx2 protein has SEQ ID NO: 2 or 4, and the polynucleotide comprises SEQ ID NO: 1 or 3.

19. The method of claim 17, wherein the antigen-free, pre-shaped allograft bone implant has a cavity suitable for receiving the pharmaceutical composition.

20. The method of claim 17, wherein the mammalian patient is a human or a domesticated animal.

21. The method of claim 20, wherein the mammalian patient is a human.
